(19) United States
(12) Patent Application Publication
(10) Pub. No.: US 2022/0110781 A1
(43) Pub. Date: Apr. 14, 2022

(54) NOSE PLUG TO ADDRESS SNORING

(71) Applicant: Mark Givler, Sugar Grove, IL (US)

(72) Inventor: Mark Givler, Sugar Grove, IL (US)

(21) Appl. No.: 17/495,486

(22) Filed: Oct. 6, 2021

Related U.S. Application Data
(60) Provisional application No. 63/090,897, filed on Oct. 13, 2020.

(51) Int. Cl. A61F 5/56

(52) U.S. Cl. CPC A61F 5/56

(57) ABSTRACT

A nose plug for the prevention of snoring and a method of forming the same. The nose plug may include a compressible section configured to be compressed and inserted into a nostril such that, once expanded, it creates an airtight seal with an inner wall of the nostril. Substantially half of volume of the compressible section may be inserted within the nostril. The nose plug may also include a second section attached to the compressible section, the second section comprising a lobe that remains outside the nostril to aid in extraction. The second section may be composed of the same material as the first material. Alternatively, the second section may be composed a material that is more elastic than the compressible section and may be bonded to the compressible section.

### CROSS REFERENCE TO RELATED APPLICATIONS

This application claims benefit to U.S. Provisional Application No. 63/090,897 entitled “Nose Plug to Address Snoring,” which was filed on Oct. 13, 2020. The full disclosure of this application is incorporated herein by reference.

### TECHNICAL FIELD

The present disclosure generally relates to nose plugs, and, more particularly, polymeric foam nose plugs having a particular functional shape such that they block the flow of air to the lungs through one or more nostrils to prevent snoring.

### BACKGROUND

Snoring is a condition characterized by rough, loud, rattling breathing or inspiratory noise during sleep or deep coma. The characteristic snoring noise is produced by vibration of the soft palate (the soft tissue in the roof of the mouth near the throat) or vocal chords by inhaled or exhaled air. As the soft palate vibrates, the lips, cheeks, and nostrils may also vibrate, making the snoring louder.

Snoring can be caused by underlying physical or disease conditions that restrict air passages and force the patient to breathe with exaggerated force to move air through narrowed nasal passages. Chronic snoring can be the result of obstruction of larynx, upper airways, a deviated nasal septum, and blockages to the nose and nasal passages. Temporary snoring, or a sudden onset of snoring can be the result of stuffy and swollen mucus membranes, as with a cold or hay fever, or a nasal polyp.

### SUMMARY

A nose plug for the prevention of snoring and a method of forming the same is described herein. The nose plug may include a compressible section configured to be compressed and inserted into a nostril such that, once expanded, it creates an airtight seal with an inner wall of the nostril. Substantially half of a volume of the compressible section may be inserted within the nostril. The nose plug may also include a second section attached to the compressible section, the second section comprising a lobe that may remain outside the nostril to aid in extraction. The second section may be composed of the same material as the first material. Alternatively, the second section may be composed a material that is more elastic than the compressible section and may be bonded to the compressible section.

### BRIEF DESCRIPTION OF THE DRAWINGS

The drawings described below are for illustration purposes only. The drawings are not intended to limit the scope of the present disclosure.

FIG. 1 is a three-dimensional view of a first nose plug;

FIGS. 2A-2B are cross-sectional views of the first nose plug inserted into a nostril;

FIG. 3 is a cross-sectional view of a second nose plug having a first section and a second section;

FIG. 4 is a three-dimensional drawing of a third nose plug;

FIGS. 5A-5B are cross-sectional views of the third nose plug inserted into a nostril; and

FIG. 6 is a cross-sectional view of a fourth nose plug having a first section and a second section.

### DETAILED DESCRIPTION

The present disclosure relates generally to a nose plug that stops the air flow to the lungs via one or more nostrils and is more particularly concerned with polymeric foam of the roll-down type.

A number of methods and devices have been developed to reduce or eliminate snoring. Some devices are external to the patient and can include, buzzer systems and alarms that wake the patient. Special pillows, neck collars, chin braces and head straps have also been tested in an effort to control snoring. When snoring is caused by serious deformity, surgery has been performed to remove anatomical obstructions, such as removing tonsils, or correcting a deviated septum. Occasionally, a procedure called UPPP (Uvulopalatopharyngoplasty) is recommended. This procedure acts like an internal facelift, tightening loose tissue. However, the success rate is typically only 50%. Laser surgery to correct airway defects is also available in some cases. Other remedies include herbal potions and medications such as decongestants and anti-histamines. Diet and lifestyle changes may also reduce snoring to some degree.

Anatomical deformities in the airway such as large tonsils and adenoids, excessive length of the soft palate, or broken or crooked nose, can also diminish the airway size. Fat deposits around the upper airway, as found in obesity, can make the airway smaller. Low muscle tone in the muscles of the tongue and throat, or medications and foods (such as alcohol) that relax these muscles also increase snoring.

Various dental devices have been developed that keep the mouth open, the tongue depressed, or nasal passages open. These dental devices can be expensive custom-fit, or inexpensive over the counter mouth pieces. Adhesive nasal strips, which are applied externally to either side of the nose, have been developed. While these strips may dilate the nasal passages to small degree, they do not work well in patients with anatomical deformities or obstructions in the nose. Air masks that force pressurized air into the mouth and lungs are available. These devices can be cumbersome, unsightly, painful, or expensive, and the patient may abandon these approaches in short time.

Conventional products focus on restricting air flow through the nasal cavity, past the soft pallet and the uvula, which in turn reduces vibrations that cause snoring. In other words, conventional products reduce, rather than eliminate, snoring. In contrast, the object of the present disclosure is to eliminate air flow via the nostril through the nasal cavity and into the lungs altogether. Utilizing a natural reflex of the human body to direct breathing through the mouth when air flow to the nose is cut off, the nose plugs disclosed herein may reduce or eliminate vibrations that are caused by turbulent air flow through the nasal cavity past the soft pallet and the uvula, thereby preventing snoring.

The disclosed nose plugs may have unique shapes to optimally fit within the typical shapes of the human nostril. The nose plugs may be made of any appropriate flexible and/or compressible material known in the art. In an example, the nose plugs may be made of a flexible polyurethane foam. In another example, the nose plugs may be made of one or more of a soft plastic, silicone, and rubber. The nose plugs may have antibacterial additives incorporated in the production process. The nose plugs may have a smooth texture to increase comfort when inserted into the human nose. In an example, the nose plugs may be disposable. In another example, the nose plugs may be reusable.

In an example, the nose plugs described herein may be made from one or more polymeric foam compositions typically used in the art of roll-down ear plugs and may be formed using conventional techniques. There are many resilient polymeric foam compositions which may be formulated by those of skill in the art to meet the viscoelastic and relatively elastic recovery property requirements for the nose plugs. By suitable selections of plasticizers, blowing agents, ingredient concentrations and blowing regimens, all of which is within the skill of the art, different foam formulations having the physical property requirements of the nose plugs may be achieved.

Polyurethane foam compositions are generally preferred as the materials for the nose plugs due to their formulation flexibility, easy processing characteristics and economics. Of these, polyether polyurethane foams may be preferred due to the generally soft surface and feel of resilience in the resulting nose plugs. Polyether polyurethane foam compositions based on polyurethane prepolymers blended with latex modifiers have been found to be useful in the art. Such polyether polyurethane prepolymers are currently available from Dow Polyurethanes under the HYPOL® brand name. Suitable latex modifiers are available from Dow, Inc. under the UCAR® brand name and the RHOPLEX® brand name. Viscoelastic foam compositions whose recovery times are temperature dependent may be produced from such latex modified polyurethane compositions. One or more parts of the nose plugs may also be made from polyvinylchloride foam compositions due to their formulation flexibility, easy processing characteristics, and economics. Polyvinylchloride foam compositions generally have stronger cell walls and tend to be more amenable to reuse and washing.

In an example, the nose plugs may have a recovery time of at least 1 second from 60% compression to 40% compression thereof. Unless otherwise stipulated herein, the recovery times mentioned herein may be taken at about room temperature, meaning approximately 21-23° C. (70-72° C.). Where the polymeric foam composition employed for the nose plugs is such that the recovery rate thereof is not markedly inversely temperature dependent, the maximum recovery time of the nose plugs may not exceed 60 seconds and may reside within a range of between 10 and 60 seconds.

The polymeric foam composition employed for the nose plugs may be of a character such that the recovery time thereof is markedly and inversely temperature dependent. Where this condition is met, the 60% to 40% compression recovery time of the nose plugs at room temperature 21-23° C. (70-72° F.) may be greater than 60 seconds. The recovery time at body temperature (e.g., approximately 35.6° C. (96° F.)) may be substantially less than the recovery time at room temperature (e.g., no greater than 90 seconds).

In an example, the material used to form the nose plugs may be mixed with an antimicrobial solution before curing. In another example, the surfaces of the nose plugs may be coated with the antimicrobial treatment, which may be sprayed onto the surfaces using a solution and/or may be applied using wipes soaked in such a solution. In an example, the antimicrobial treatment solution may contain 30% to 50% isopropyl alcohol and 50% to 70% percent antimicrobial treatment substance, which may be a silane quaternary ammonium salt having an unreacted organofunctional silane. If the antimicrobial treatment solution is applied by spraying, the solution may include 50% percent isopropyl alcohol and 50% of the unreacted antimicrobial treatment substance. If the solution is applied using wipes, the solution may include 30% isopropyl alcohol and 70% of the unreacted antimicrobial treatment substance.

The above described silane quaternary ammonium salt may be used as the antimicrobial treatment because it is an organofunctional silane antimicrobial treatment substance that is substantially free from arsenic, silver, tin, heavy metals and polychlorinated phenols; copper; or a silver-ion emitter. In addition, the silane quaternary ammonium not only eliminates bacteria on contact, but it may remain on the treated surfaces and kill any bacteria subsequently contacting these surfaces. One or more coloring agents and other additives may also be mixed into the polymer before curing.

The nose plugs may not need to be compressed greatly below the size of the nostrils into which they are to be inserted in order to assure adequate time in which to insert them and the user need not rush the insertion step in order to assure adequate depth of insertion thereof. In use, the nose plug may initially be rolled down to below the size of the nostril into which it is to be inserted. The relatively slow recovery viscoelastic nose plug may recover to contact the enclosing nostril wall and may develop a stable airtight seal therewith.

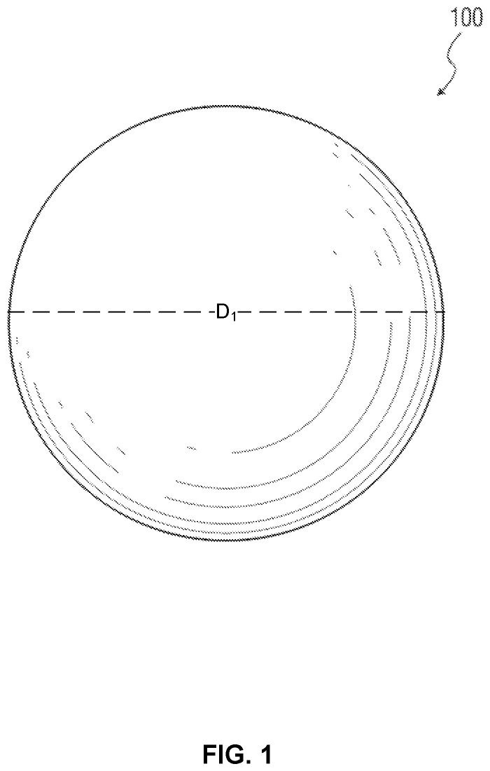

Referring now to FIG. 1, a three-dimensional view of a first nose plug 100 is shown. The first nose plug 100 may include a compressible section that is substantially spherical in shape and may have a diameter Di ranging from approximately 0.125 in to approximately 2.0 in.

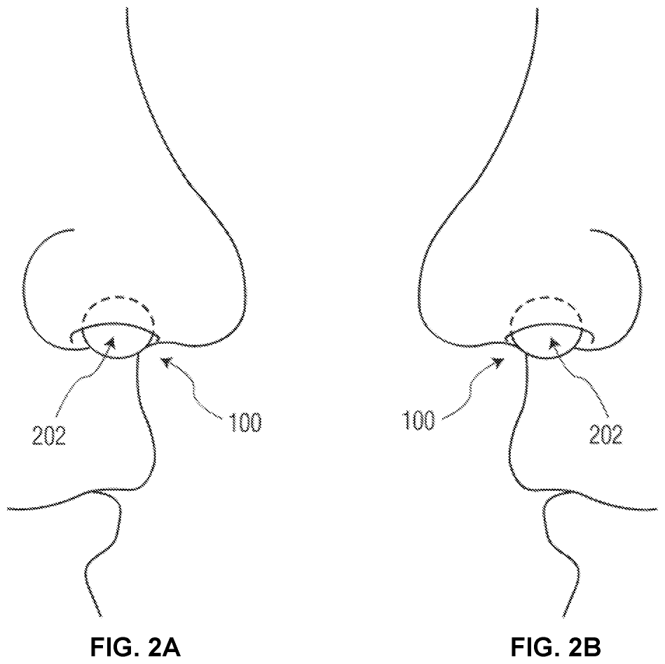

Referring now to FIGS. 2A-2B, cross-sectional views of the first nose plug 100 inserted into a nostril is shown. As shown, the first nose plug 100 may fit into the nostril such that approximately one half of the volume of the compressible section is inserted. In other words, a mid-point 202 of the first nose plug 100 may be substantially aligned with the opening of the nostril. It should be clear, however, that the first nose plug 100 may be designed such that more or fewer of its volume may be inserted into the nostril.

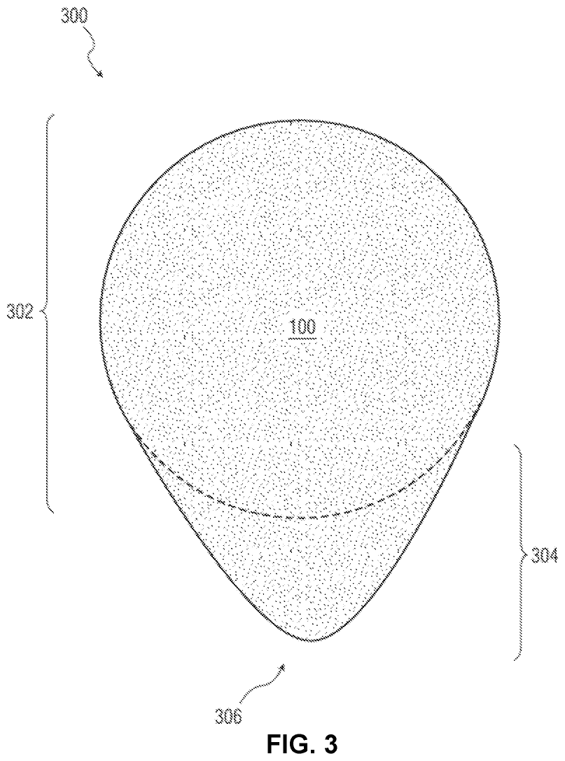

Referring now to FIG. 3, a cross-sectional view of a second nose plug 300 is shown. The second nose plug 300 may be composed of two sections. A compressible first section 302 may be similar to the first nose plug 100 described above and substantially spherical in shape. The second section 304 may be a lobe to assist in the extract of the second nose plug 300 from the nostril. The second section 304 may remain outside (fully or partially) of the nostril during use.

The second section 304 may start from a point that is in relation to approximately 75% of the circumference of the first nose plug 100. In an example, the second section 304 may be in the general shape of a cone (or cylinder) and may protrude from the first section 302 at an angle ranging from approximately 1 degree to approximately 7 degrees. The second section 304 may have a length extending from the first section 302 of approximately ⅜ in. An endpoint 306 of the second section 304 may have a diameter ranging from approximately 3/16 in to approximately 0.5 in. It should be understood, however, that the second section 304 may comprise any suitable dimensions and/or attributes, according to the particular implementation.

In an example, the second section 304 may be composed of the same material as the first nose plug 100 and may be formed at the same time as the first nose plug 100, using the techniques described above, via a single mold.

In another example, the second section 304 may be composed of a different material. The second section 304 may be more elastic in nature than the first section 302. The relatively elastic second section 304 may have a recovery time from approximately 60% compression to 40% compression which is no greater than about 50% of the recovery time of the first nose plug 100 and may not exceed 5 seconds, although other compression ratios and/or recovery times may also be acceptable.

Fixation of the first section 302 to the second section 304 may be achieved in any suitable manner, such as by means of a suitable adhesive, solvent welding or thermal welding of the respective sections. The fixation may be achieved by direct bonding of the two polymeric foam sections together either by molding of two serially deposited polymeric foam purchaser compositions in a mold or by manufacture of a two-layer sheet of the nose plug foam materials followed by die cutting of earplugs therefrom. By directly bonding the first section 302 and the second section 304 together, in other words, by avoiding the interposition of glues, adhesives, thermoplastic sheets and the like between said sections, there is generally avoided a parting line which may feel distinctly different from the polymeric foam sections themselves and this avoidance is desirable from the standpoint of maximizing the sensed comfort of the second nose plug 300 during its use. The molding technique outlined above may result in direct bonding of the first section 302 to the second section 304.

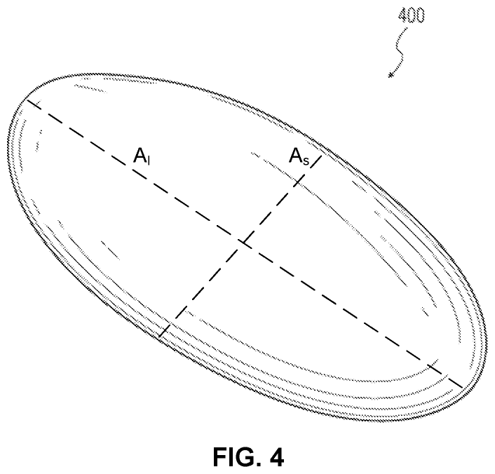

Referring now to FIG. 4, a three-dimensional drawing of a third nose plug 400 is shown. The third nose plug 400 may have a compressible section that is substantial elliptical in shape and may have a short axis Aₛ and a long axis A₁. The long axis A₁ may have a length ranging from approximately 0.125 in to approximately 2.0 in., although any suitable length according to the particular implementation may be utilized.

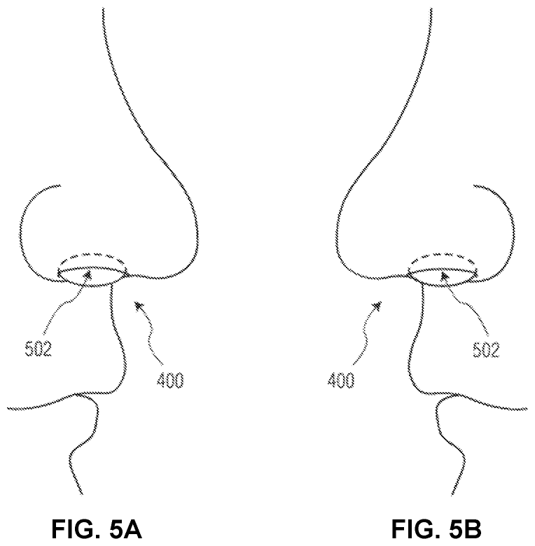

Referring now to FIGS. 5A-5B, cross-sectional views of the third nose plug 400 inserted into a nostril is shown. As shown, the third nose plug 400 may fit into the nostril such that approximately one half of the volume of the compressible section is inserted. In other words, a mid-point 502 of the third nose plug 400 may be substantially aligned with the opening of the nostril, although more or less of its volume may be inserted.

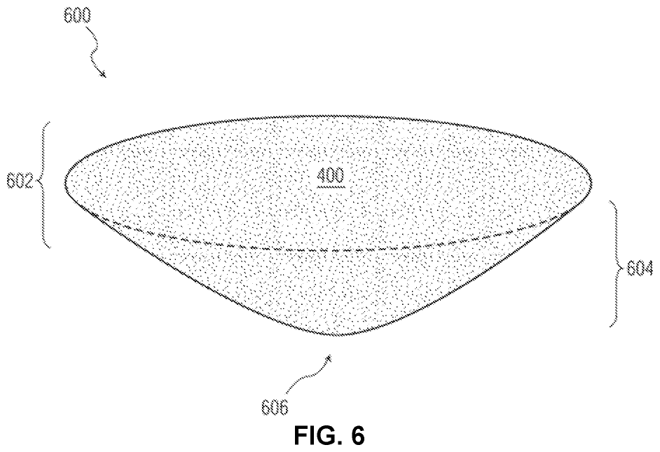

Referring now to FIG. 6, a cross-sectional view of a fourth nose plug 600 is shown. The fourth nose plug 600 may be composed of two sections. A compressible first section 602 may be similar to the third nose plug 400 described above and may substantially elliptical in shape. The second section 604 may be a lobe to assist in the extract of the fourth nose plug 600 from the nostril. The second section 604 may remain (fully or partially) outside of the nostril during use.

The second section 604 may extended from a line that is approximately 75% of the circumference of the third nose plug 400 in relation to the long axis. In an example, the second section 604 may be in the general shape of a cone (or cylinder) and may protrude from the first section 602 at an angle ranging from approximately 1 degree to approximately 7 degrees. The second section 604 may have a length extending from the first section 602 of approximately ⅜ in. An endpoint 606 of the second section 604 may have a diameter ranging from approximately 3/16 in to approximately 0.5 in. It should be understood, however, that the second section 604 may comprise any suitable dimensions and/or attributes, according to the particular implementation.

In an example, the second section 604 may be composed of the same material as the third nose plug 400 and may be formed at the same time as the third nose plug 400, using the techniques described above, via a single mold.

In another example, the second section 604 may be composed of a different material. The second section 604 may be more elastic in nature than the first section 602. The relatively elastic second section 604 may have a recovery time from approximately 60% compression to 40% compression which is no greater than about 50% of the recovery time of the third nose plug 400 and may not exceed 5 seconds, although other compression ratios and/or recovery times may also be acceptable.

In an example, fixation of the first section 602 to the second section 604 may be achieved in any suitable manner, such as by means of a suitable adhesive, solvent welding or thermal welding of the respective sections. The fixation may be achieved by direct bonding of the two polymeric foam sections together either by molding of two serially deposited polymeric foam purchaser compositions in a mold or by manufacture of a two-layer sheet of the nose plug foam materials followed by die cutting of earplugs therefrom. By directly bonding the first section 602 and the second section 604 together, in other words, by avoiding the interposition of glues, adhesives, thermoplastic sheets and the like between said sections, there is generally avoided a parting line which may feel distinctly different from the polymeric foam sections themselves and this avoidance is desirable from the standpoint of maximizing the sensed comfort of the fourth nose plug 600 during its use. The molding technique outlined above may result in direct bonding of the first section 602 to the second section 604.

Although features and elements are described above in particular combinations, one of ordinary skill in the art will appreciate that each feature or element can be used alone or in any combination with the other features and elements.

1. A nose plug for the prevention of snoring comprising:
a compressible section configured to be compressed and inserted into a nostril such that, once expanded, it creates an airtight seal with an inner wall of the nostril.

2. The nose plug of claim 1, wherein the compressible section comprises a polymeric foam.

3. The nose plug of claim 2, wherein the polymeric foam comprises an antimicrobial solution.

4. The nose plug of claim 1, wherein the compressible section is substantially spherical in shape.

5. The nose plug of claim 4, wherein the compressible section has a diameter ranging from approximately 0.125 in to approximately 2.0 in.

6. The nose plug of claim 1, wherein the compressible section is substantially elliptical in shape.

7. The nose plug of claim 6, wherein the compressible section has a short axis and a long axis, the long axis having a length ranging from approximately 0.125 in to approximately 2.0 in.

8. The nose plug of claim 1, further comprising:
a second section attached to the compressible section, the second section comprising a lobe that remains outside the nostril to aid in extraction.

9. The nose plug of claim 8, wherein the second section is substantially conical in shape.

10. The nose plug of claim 8, wherein the second section comprises one or more of a material that is substantially similar in composition to the compressible section and a more elastic material than the compressible section that is directly bonded to the compressible section.

11. A method of forming a nose plug for the prevention of snoring comprising:
forming a compressible section configured to be compressed and inserted into a nostril, such that, once expanded, it creates an airtight seal with an inner wall of the nostril.

12. The method of claim 11, wherein the compressible section comprises a polymeric foam.

13. The method of claim 12, wherein the polymeric foam comprises an antimicrobial solution.

14. The method of claim 11, wherein the compressible section is substantially spherical in shape.

15. The method of claim 14, wherein the compressible section has a diameter ranging from approximately 0.125 in to approximately 2.0 in.

16. The method of claim 11, wherein the compressible section is substantially elliptical in shape.

17. The method of claim 16, wherein the compressible section has a short axis and a long axis, the long axis having a length ranging from approximately 0.125 in to approximately 2.0 in.

18. The method of claim 11, further comprising:
forming a second section attached to the compressible section, the second section comprising a lobe that remains outside the nostril to aid in extraction.

19. The method of claim 18, wherein the second section is substantially conical in shape.

20. The method of claim 19, wherein the second section comprises one or more of a material that is substantially similar in composition to the compressible section and a more elastic material than the compressible section that is directly bonded to the compressible section.
